(19) United States
(12) Patent Application Publication
(10) Pub. No.: US 2011/0177068 A1
(43) Pub. Date: Jul. 21, 2011

(54) PHARMACEUTICAL PREPARATION COMPRISING AN ANTIBODY AGAINST THE EGF RECEPTOR

(76) Inventors: Robert MUELLER, Darmstadt (DE); Hanns-Christian Mahler, Wiesbaden (DE)

(21) Appl. No.: 13/070,952

(22) Filed: Mar. 24, 2011

Related U.S. Application Data
(63) Continuation of application No. 10/996,597, filed on Nov. 26, 2004, now abandoned.

(30) Foreign Application Priority Data
Nov. 26, 2003 (DE) 1035521.0

(51) Int. Cl. A61K 39/395; A61P 35/00

(52) U.S. Cl. 424/133.1; 424/172.1

(57) ABSTRACT

The invention relates to an aqueous pharmaceutical preparation comprising an antibody against endothelial growth factor receptor (EGF receptor). The preparation has increased storage stability, even at elevated temperatures, and can be used for the treatment of tumours.

This application claims the benefit of the filing date of U.S. Provisional Application Ser. No. 60/543,287 filed Feb. 11, 2004 which is incorporated by reference herein.

The present invention relates to a stable pharmaceutical preparation comprising an antibody which is directed against the epidermal growth factor receptor (EGFR), and to the preparation and use thereof.

Various in vitro and in vivo studies have shown that blockage of EGFR by antibodies acts against tumours at various levels, for example by inhibiting cancer cell proliferation, reducing tumour-mediated angiogenesis, inducting cancer cell apoptosis and increasing the toxic effects of radiotherapy and conventional chemotherapy

MAB c225 (INN: cetuximab) is a clinically proven antibody which binds to the EGF receptor. Cetuximab is a chimeric antibody whose variable regions are of murine origin and whose constant regions are of human origin. Cetuximab was described for the first time by Naramura et al., Cancer Immunol. Immunotherapy 1993, 37: 343-349 and in WO 96/40210 A1.

MAB 425 is an originally murine antibody which is directed against EGFR which is overexpressed in tumour cells, in particular A431 carcinoma cells. Its humanised and chimeric forms are disclosed, for example, in EP 0 531 472 A1; Kettleborough et al., Protein Engineering 1991, 4: 773-783; Bier et al., Cancer Chemother. Pharmacol. 2001, 47: 519-524; Bier et al., Cancer Immunol. Immunother. 1998, 46: 167-173. EMD 72000 (h425) is a form of MAB 425 which is in clinical phase I/II and whose constant region is composed of a κ and a human γ-1 chain.

Human anti-EGFR antibodies can be provided by XenoMouse technology, as described in WO 91/10741 A1, WO 94/02602 A1 and WO 96/33735 A1. A specific antibody which has been produced by this technology and is currently undergoing clinical trials is ABX-EGF (Abgenix, Crit. Rev. Oncol. Hematol. 2001, 38: 17-23; Cancer Research 1999, 59: 1236-43).

Further antibodies directed against EGFR are described, for example, in EP 0 586 002 B1 and in J. Natl. Cancer Inst. 1993, 85: 27-33 (MAB 528).

Like other antibodies, anti-EGFR antibodies are also applied parenterally as a solution for therapeutic use. A particular problem of solutions comprising these antibodies is their tendency toward aggregation and the formation of protein multimers. In the case of reducible multimers, this can be attributed to unintentional intermolecular disulfide bridge formation through interaction between approaching moieties. Hydrophobic interactions and the consequent formation of non-reducible multimers are also possible. Furthermore, deamidation reactions, which subsequently result in protein degradation reactions, also occur. The denaturing reactions described occur, in particular, on storage at elevated temperature or during shear stresses, as occur, for example, during transport.

As a consequence of the said aggregation tendency, product precipitation occurs during storage of antibody solutions, meaning that reproducible withdrawal from the container containing the solution is doubtful. In addition, emboli can occur on parenteral administration of particle-containing solution. This has the consequence that the administration of the anti-EGFR antibodies to the patient in the requisite dose in each case is not always guaranteed in a reproducible manner by means of antibody solutions, and the administration cannot take place with the requisite reliability.

Although filtration before injection enables the aggregates to be held back, this method, however, comprises an additional step and is therefore complex and not very suitable for clinical practice. In addition, the problem of dose reproducibility remains unsolved, since an unknown fraction of antibodies is in each case separated off from the solution, and particle formation after filtration continues to represent a safety risk.

A common method for the stabilisation of monoclonal antibodies is freeze-drying of solutions comprising antibodies and auxiliaries. However, lyophilisation is very time- and energy-consuming and thus expensive. In addition, the lyophilisate must first be reconstituted before administration.

EP 0 073 371 describes preparations for intravenous administration which comprise immunoglobulins which have a pH of from 3.5 to 5.0 for stabilisation. However, such low pH values result in undesired incompatibility reactions at the injection point.

U.S. Pat. No. 6,171,586 B1 discloses the use of an acetate buffer pH 4.48 to 5.5, a surfactant and a polyol in an aqueous formulation of antibodies, with NaCl for isotonicity modification being excluded. Owing to the lack of isotonicity modification, incompatibility reactions at the injection point may likewise occur.

As examples of further formulations comprising specific antibodies, reference may be made at this point to EP 0 280 358, EP 0 170 983 and U.S. Pat. No. 5,945,098.

Of these, EP 0 280 358 describes the addition of dextran to an antibody solution for stabilisation against certain hormones, where stability was achieved over nine months.

EP 0 170 983 describes the stabilisation of a thermolabile monoclonal antibody by heating together with hydrolysed ovalbumin, as a result of which the antibody was still stable after storage for 7 days at 45° C. However, the addition of proteins of other species to formulations intended for parenteral administration is undesired owing to the problems associated therewith, in particular their possible antigeneity.

U.S. Pat. No. 5,945,098 discloses the use of glycine, polysorbate 80 and polyethylene glycol for the stabilisation of an aqueous solution of immunoglobulin G.

DE 10133394 A1 discloses the use of a phosphate buffer in the range from pH 6 to pH 8 and a polyoxyethylene sorbitan fatty acid ester for the stabilisation of an aqueous solution of the antibody cetuximab. Although this significantly reduces the formation of visible aggregates, the chemical stability, in particular under stress conditions, is significantly impaired. Furthermore, the formulation does not exhibit stability to (extreme) thermal stress, for example long-term storage at 40° C.

The object of the invention was to find an aqueous formulation specifically for the antibodies directed against EGFR which is suitable for parenteral administration, is well tolerated and is stable on storage at room temperature over at least 24 months. The storage stability should also be retained in the case of shear forces acting during transport and under modified climatic conditions, in particular at elevated temperature and atmospheric humidity. Furthermore, the formulation should have a simple structure and should not comprise any auxiliaries which are dubious from a toxicological point of view.

Surprisingly, a formulation which meets these requirements has been found in the form of a solution which, in addition to an antibody directed against epidermal growth factor (anti-EGFR antibody), comprises a buffer, an amino acid and a surfactant. The present invention therefore relates to an aqueous pharmaceutical preparation which, in addition to an anti-EGFR antibody, comprises a buffer, an amino acid and a surfactant.

For the purposes of the invention, an aqueous preparation is one in which at least some of the solvent present consists of water. Further solvent constituents which may be present are all solvents which are suitable for parenteral use, in particular alcohols, such as, for example, ethanol, propanol, propanediol or glycerol. The aqueous preparation preferably comprises, as solvent, water or ethanol/water mixtures; the solvent particularly preferably consists of water.

The antibody present can be any anti-EGFR antibody, in particular the murine, humanised or chimeric antibodies mentioned at the outset and the human anti-EGFR antibodies which have been and can be prepared by means of the said XenoMouse technology. Preference is given to the anti-EGFR antibody cetuximab dr EMD 72000 or one of the murine, humanised or chimeric antibody analogues corresponding thereto. Particular preference is given to aqueous preparations which comprise cetuximab or EMD 72000 as antibody.

The anti-EGFR antibody may be present in the, formulation according to the invention in a concentration of from 0.1 mg/ml to 50 mg/ml, preferably from 2 mg/ml to 10 mg/ml, particularly preferably about 5 mg/ml.

Buffers which can be employed are basically all physiologically tolerated substances which are suitable for setting the desired pH, such as, for example, citrate salts, acetate salts, histidine salts, succinate salts, malate salts, phosphate salts or lactate salts, and/or the respective free acids or bases thereof, as well as mixtures of the various salts and/or acids or bases thereof. The buffer preferably consists of one or more citrate salt(s), acetate salt(s), histidine salt(s), succinate salt(s), malate salt(s), phosphate salt(s) or lactate salt(s) and/or the respective free acid(s) or base(s) thereof or a mixture of one or more of the various salts and/or the acid(s) or base(s) thereof. The term mixture here covers both mixtures of different salts of the same acid, such as, for example, mixtures of different citrate salts, and mixtures of salts of different acids, such as, for example, mixtures of citrate and acetate salts. The buffer preferably consists of one or more citrate salt(s) and/or the free acid thereof (for example citric acid, citric acid monohydrate, trisodium citrate dihydrate, tripotassium citrate monohydrate), acetate salt(s) and/or the free acid thereof (for example acetic acid, sodium acetate, sodium acetate trihydrate) or L-histidine and/or an acid-addition salt thereof, such as, for example, L-histidine monohydro-chloride monohydrate. The preparation according to the invention advantageously comprises the buffer in a concentration of from 10 to 100 mmol/l, preferably from 2 to 20 mmol/l, particularly preferably about 10 mmol/l.

The pH of the preparation is in the range from 5.0 to 6.0, preferably from 5.2 to 5.8, particularly preferably about 5.5.

The preparation according to the invention is physiologically well tolerated, can be prepared easily, can be dispensed precisely and is stable with respect to assay, decomposition products and aggregates over the duration of storage, during repeated freezing and thawing processes and mechanical stress. It is stable on storage over a period of at least 3 months to a period of 4 years at refrigerator temperature (2-8° C.). Surprisingly, the preparation according to the invention is also stable on storage over a period of up to 2 years at elevated temperatures and higher atmospheric humidity levels, for example at a temperature of 25° C. and 60% relative atmospheric humidity, or over a period of 3 months at a temperature of 40° C. and 75% relative atmospheric humidity.

The amino acid present in the preparation can be basic amino acids, such as, for example, arginine, histidine, ornithine, lysine, or neutral amino acids, such as, for example, glycine, methionine, isoleucine, leucine and alanine, or aromatic amino acids, such as, for example, phenylalanine, tyrosine or tryptophan. Basic amino acids are preferably employed in the form of their inorganic salts (advantageously in the form of the hydrochloric acid salts, i.e. as amino acid hydrochlorides). The amino acid employed is preferably in each case the L-form. The amino acid present in the preparation according to the invention is particularly preferably L-arginine, glycine or L-methionine.

The preparation comprises the amino acid in a concentration of from 2 to 200 mmol/l, preferably from 50 to 150 mmol/l, particularly preferably about 100 mmol/l.

Surfactants which can be employed are all surfactants usually used in pharmaceutical preparations, preferably polyethylene sorbitan fatty acid esters and polyoxyethylenetpolyoxypropylene copolymers. Polyethylene sorbitan fatty acid esters are also known under the trade name Tween. Suitable polyethylene sorbitan fatty acid esters are, in particular, polyoxyethylene (20) sorbitan monolaurate, polyoxyethylene (20) sorbitan monopalmitate and polyoxyethylene (20) sorbitan monostearate. Preference is given to polyoxyethylene (20) sorbitan monolaurate and polyoxyethylene (20) sorbitan monooleate, particular preferewnce being given to polyoxyethylene (20) sorbitan monooleate. Polyoxyethylene-polyoxypropylene copolymers are also known under the trade name Poloxamer.

A particularly preferred polyoxyethylene-polyoxypropylene copolymer is Poloxamer 407 (CAS 9003-11-6).

The surfactants may be present in the formulation in a concentration of from 0.001% to 1.0% by weight. If polyoxyethylene sorbitan fatty acid esters are present as surfactants, these are preferably present in an amount of from 0.005 to 0.1% by weight, particularly preferably in an amount of about 0.01% by weight. If polyoxyethylene-polyoxypropylene copolymers are present, these are preferably present in an amount of from 0.01 to 0.5% by weight, particularly preferably about 0.1% by weight.

In order to increase the tolerability on parenteral administration, the osmolality is preferably in the isotonic range, i.e. at an osmolality of from about 250 to 350 mOsmol/kg. The preparation can then be administered directly intravenously, intraarterially and also subcutaneously substantially without pain.

According to an advantageous embodiment, the preparation according to the invention therefore additionally comprises an isotonicity modifier, preferably a physiologically tolerated salt, such as, for example, sodium chloride or potassium chloride, or a physiologically tolerated polyol, such as, for example, glucose or glycerol, in a concentration necessary for isotonicity modification. The invention therefore furthermore relates to an aqueous preparation comprising an anti-EGFR antibody, a buffer, an amino acid, a surfactant and an isotonicity modifier in a concentration necessary for isotonicity modification. Sodium chloride is particularly preferably present as isotonicity modifier.

In addition, the solutions according to the invention may comprise further physiologically tolerated auxiliaries, such as, for example, antioxidants, such as ascorbic acid or glutathione, preservatives, such as phenol, m-cresol, methyl- or propylparaben, chlorobutanol, thiomersal or benzalkonium chloride, polyethylene glycols (PEG), such as PEG 400, PEG 3000, 3350, 4000 or 6000, disaccharides, such as trehalose or saccharose, or cyclodextrins, such as hydroxypropyl-β-cyclodextrin, sulfobutylethyl-β-cyclodextrin, α-cyclodextrin or γ-cyclodextrin.

According to a particularly advantageous embodiment of the invention, the aqueous preparation comprises about 5 mg/ml of cetuximab or EMD 72000, about 10 mmol/l of citrate or histidine buffer having a pH of about 5.5, about 100 mmol/ l of glycine, arginine or L-methionine, about 100 mmol/l of sodium chloride and about 0.01% of polyoxyethylene (20) sorbitan monooleate.

The aqueous preparation can be prepared by adding the said auxiliaries to a solution comprising the anti-EGFR antibody. To this end, defined volumes of stock solutions comprising the said further auxiliaries in defined concentration are advantageously added to a solution having a defined concentration of the anti-EGFR antibody, as obtained from its preparation, and the mixture is, if desired, diluted to the pre-calculated concentration with water or buffer solution. Alternatively, the auxiliaries can also be added as solids to the starting solution comprising the anti-EGFR antibody. If the anti-EGFR antibody is in the form of a solid, for example in the form of a lyophilisate, the preparation according to the invention can be prepared by firstly dissolving the respective antibodies in water or an aqueous solution comprising one or more of the further auxiliaries, and subsequently adding the amounts required in each case of stock solutions comprising the further auxiliaries, the further auxiliaries in solid form and/or water. The anti-EGFR antibody can advantageously also be dissolved directly in a solution comprising all further auxiliaries.

One or more of the auxiliaries present in the preparation according to the invention may advantageously already have been added during or at the end of the process for the preparation of the particular EGFR antibody. This can preferably be carried out by dissolving the anti-EGFR antibody directly in an aqueous solution comprising one, more than one or all of the further auxiliaries in the final step of the purification carried out after its preparation. In order to prepare the preparation, the respective further ingredient(s) then need only be added in a smaller amount in each case and/or not added at all. It is particularly preferred for the respective ingredient to be dissolved directly in an aqueous solution comprising all further auxiliaries in the final step of the purification carried out after its preparation.

If the solution comprising the respective antibody and the auxiliaries does not yet have the desired pH, this is set by addition of an acid or base, preferably using the acid or base already present in the buffer system. This is followed by sterile filtration.

The aqueous preparation according to the invention can advantageously be employed for the treatment of tumour diseases.

The examples explain the invention without being restricted thereto.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, ,all temperatures are set forth uncorrected in degrees Celsius and, all parts and percentages are by weight, unless otherwise indicated.

### EXAMPLE 1 (COMPARATIVE EXAMPLE 1)

Aqueous solution comprising:

2 mg/ml of cetuximab

10 mmol/l of sodium phosphate buffer pH 7.2

145 mmol/l of sodium chloride

The preparation is carried out by mixing defined volumes of aqueous solutions comprising the respective auxiliaries in defined concentration. The following solutions are used:

Solution A (active ingredient solution) comprising:

18 mg/ml of cetuximab

10 mmol/l of sodium phosphate buffer pH 7.2

(consisting of 2.07 g/l of disodium hydrogenphosphate 7-hydrate and

0.31 g/I of sodium dihydrogenphosphate monohydrate)

145 mmol/l of sodium chloride

(The solution is obtained by re-buffering the active ingredient against solution B with the aid of tangential flow filtration in the final step of the active-ingredient purification taking place after preparation thereof.)

Solution B (buffer/salt solution)

Corresponds to solution A, but comprises no active ingredient.

For the preparation of Comparative Solution 1, 1.11 parts by volume of solution A and 8.89 parts by volume of solution B are combined with one another.

The solution prepared is filtered using a sterile filter before transfer and transferred into injection vials. The injection vials are subsequently sealed with stoppers and crimped.

### EXAMPLE 2 (COMPARATIVE EXAMPLE 2)

Aqueous solution conprising:

2 mg/ml of cetuximab

10 mmol/l of sodium phosphate buffer pH 7.2

145 mmol/l of sodium chloride

0.01% by weight of polyoxyethylene (20) sorbitan monooleate

The preparation is carried out by mixing defined. volumes of aqueous solutions comprising the respective ingredients in defined concentration. Besides solution A, the following solution is used.

Solution C (buffer/salt solution comprising polyoxyethylene (20) sorbitan monooleate)

Corresponds to solution B, but additionally comprises 0.0125% by weight of polyoxyethylene (20) sorbitan monooleate.

For the preparation of comparative solution 2, 1.11 parts by volume of solution A and 8.89 parts volume of solution C are combined with one another.

The solution prepared is filtered using a sterile filter before transfer and transferred into injection vials. The injection vials are subsequently sealed with stoppers and crimped.

### EXAMPLE 3 (FORMULATION ACCORDING TO THE INVENTION)

5 mg/ml of cetuximab

10 mmol/l of citrate buffer pH 5.5

100 mmol/l of glycine

100 mmol/l of sodium chloride

0.01% by weight of polyoxyethylene (20) sorbitan monooleate

The preparation is carried out by mixing defined volumes of aqueous solutions comprising the respective ingredients in defined concentrations.

Solution D (active ingredient solution in a citrate buffer)

16 mg/ml of cetuximab

10 mmol/l of citrate buffer pH 5.5

(consisting of 2.1014 g/l of citric acid monohydrate)

(The solution is obtained by re-buffering the active ingredient against solution E with the aid of tangential flow filtration in the final step of the active-ingredient purification taking place after preparation thereof.)

Solution E (buffer solution):

Corresponds to solution D, but comprises no active ingredient.

Solution F (buffer/salt solution):

Corresponds to solution E, but comprises

145.5 mmol/l of glycine,

145.5 mmol/l of sodium chloride and

0.015% by weight of polyoxyethylene (20) sorbitan monooleate.

For the preparation of the formulation according to the invention, 3.125 parts by volume of solution D and 6.875 parts by volume of solution F are combined with one another.

The solution prepared is filtered using a sterile filter before transfer and transferred into injection vials. The injection vials are subsequently sealed with stoppers and crimped.

### EXAMPLE 4

The following solutions are prepared analogously to the method of the examples described above:

### EXAMPLE 4.1

solution comprising:

5 mg/ml of cetuximab

100 mmol/l of glycine

0.01% by weight of polyoxyethylene (20) sorbitan monooleate

10 mmol/l of citrate buffer pH 5.5

(consisting of 2.9410 g/l of trisodium citrate dihydrate)

### EXAMPLE 4.2

solution comprising:

5 mg/ml of cetuximab

100 mmol/l of glycine

100 mmol/l of sodium chloride

0.01% by weight of polyoxyethylene (20) sorbitan monooleate

10 mmol/l of citrate buffer pH 5.5

(consisting of 2.1014 g/l of citric acid monohydrate)

### EXAMPLE 4.3

solution comprising:

5 mg/ml of EMD 72000

100 mmol/lof glycine

100 mmol/l of sodium chloride

0.01% by weight of polyoxyethylene (20) sorbitan monooleate

10 mmol/l of citrate buffer pH 5.5

(consisting of 2.1014 g/l of citric acid monohydrate)

### EXAMPLE 4.4

solution comprising:

5 mg/ml of cetuximab

100 mmol/I of L-methionine

0.01% by weight of polyoxyethylene (20) sorbitan monooleate

10 mmol/l of citrate buffer pH 5.5

(consisting of 2.1014 g/l of citric acid monohydrate)

### EXAMPLE 4.5

solution comprising:

5 mg/ml of cetuximab

100 mmol/l of glycine

0.01% by weight of polyoxyethylene (20) sorbitan monooleate

10 mmol/l of acetate buffer pH 5.5

(consisting of 1.3608 g/l of sodium acetate trihydrate)

### EXAMPLE 4.6

solution comprising:

5 mg/ml of cetuximab

100 mmol/l of glycine

0.01% by weight of polyoxyethylene (20) sorbitan monooleate

10 mmol/l of histidine buffer pH 5.5

(consisting of 2.069 g/l of L-histidine monohydrochloride monohydrate)

### EXAMPLE 4.7

solution comptising:

5 mg/ml of cetuximab

100 mmol/l of glycine

0.01% by weight of polyoxyethylene (20) sorbitan monolaurate

10 mmol/l of citrate buffer pH 5.5

(consisting of 2.1014 g/l of citric acid monohydrate)

### EXAMPLE 4.8

solution comprising:

5 mg/ml of cetuximab

100 mmol/l of glycine

0.1% by weight of polyoxyethylene-polyoxypropylene copolymer 407

(Poloxamer 407)

10 mmol/l of citrate buffer pH 5.5

(consisting of 2.1014 g/l of citric acid monohydrate)

### EXAMPLE 5

The stability of the formulation according to the invention was tested in a stress test. To this end, vials containing the solution according to Example 3 and, for comparative purposes, vials containing solution according to Examples 1 and 2 were stored at 25° C. and 60% relative atmospheric humidity and 40° C. and 75% relative atmospheric humidity. In addition, vials containing solutions according to Examples 1, 2 and 3 were shaken for five days in a shaking apparatus at a shaking frequency of 150 min⁻¹ at room temperature and frozen three times in succession at −20° C. and subsequently thawed again at +5° C. Before storage and after defined storage times, the vials were assessed visually with direct illumination with a cold-light source, and the absorption of the solutions at 350 nm, which represents a measure of the turbidity, was determined. In order to illustrate the influence of storage or treatment, the relative turbidity was in each case calculated relative to the starting value. Furthermore, the vials were analysed by means of HPLC gel filtration with respect to the content of cetuximab, aggregates and decomposition products.

The results of the stability investigations are shown in Table 1.

TABLE 1
Summary of the stability data of the formulation according to the
invention (Example 3) and the two comparative solutions (Examples 1 and 2)

| | | | | Decom- | | Relative | |
|---|---|---|---|---|---|---|---|
| | Storage | | | position | Turbidity | turbidity | |
| Test | [time/ | Cetuximab | Aggregates | products | at λ = | at λ = | Visual |
| solution | conditions] | [%] | [%] | [%] | 350 nm | 350 nm | assessment |
| Example 1 | 0 weeks | 99.67 | 0.12 | 0.22 | 0.005 | 1.00 | Small |
| | | | | | | | particles, |
| | | | | | | | low |
| | | | | | | | number, |
| | | | | | | | clear |
| Example 1 | 8 weeks | 98.99 | 0.28 | 0.73 | 0.0081 | 1.62 | Large |
| | 25° C./60% | | | | | | particles, |
| | R.H. | | | | | | large |
| | | | | | | | number, |
| | | | | | | | clear |
| Example 1 | 8 weeks | 95.08 | 3.23 | 1.69 | 0.0235 | 4.70 | Large |
| | 40° C./75% | | | | | | particles, |
| | R.H. | | | | | | large |
| | | | | | | | number, |
| | | | | | | | clear |
| Example 1 | Shaking | 99.60 | 0.17 | 0.24 | 0.829 | 165.80 | Very large |
| | for 5 days | | | | | | particles, |
| | at 150 rpm | | | | | | very high |
| | and RT | | | | | | particle |
| | | | | | | | number, |
| | | | | | | | cloudy |
| Example 1 | 3 freeze/ | 99.68 | 0.14 | 0.18 | 0.0089 | 1.78 | Large |
| | thaw | | | | | | particles, |
| | cycles | | | | | | high |
| | between | | | | | | particle |
| | −20° C. and | | | | | | content, |
| | +5° C. | | | | | | slightly |
| | | | | | | | cloudy |
| Example 2 | 0 weeks | 99.62 | 0.18 | 0.21 | 0.0048 | 1.00 | No |
| | | | | | | | particles, |
| | | | | | | | clear |
| Example 2 | 8 weeks | 99.02 | 0.28 | 0.70 | 0.0071 | 1.48 | Small |
| | 25° C./60% | | | | | | particles, |
| | R.H. | | | | | | low |
| | | | | | | | number, |
| | | | | | | | clear |
| Example 2 | 8 weeks | 93.95 | 4.34 | 1.72 | 0.0241 | 5.02 | Small |
| | 40° C./75% | | | | | | particles, |
| | R.H. | | | | | | low |
| | | | | | | | number, |
| | | | | | | | clear |
| Example 2 | Shaking | 99.51 | 0.26 | 0.23 | 0.0075 | 1.56 | No |
| | for 5 days | | | | | | particles, |
| | at 150 rpm | | | | | | clear |
| | and RT | | | | | | |
| Example 2 | 3 freeze/ | 99.61 | 0.21 | 0.18 | 0.0064 | 1.48 | No |
| | thaw cycles | | | | | | particles, |
| | between | | | | | | clear |
| | −20° C. and | | | | | | |
| | +5° C. | | | | | | |
| Example 3 | 0 weeks | 99.72 | 0.15 | 0.14 | 0.018 | 1.00 | No |
| | | | | | | | particles, |
| | | | | | | | clear |
| Example 3 | 8 weeks | 99.38 | 0.18 | 0.44 | 0.020 | 1.08 | Small |
| | 25° C./60% | | | | | | particles, |
| | R.H. | | | | | | low |
| | | | | | | | number, |
| | | | | | | | clear |
| Example 3 | 8 weeks | 98.15 | 0.46 | 1.40 | 0.030 | 1.63 | Small |
| | 40° C./75% | | | | | | particles, |
| | R.H. | | | | | | low |
| | | | | | | | number, |
| | | | | | | | clear |
| Example 3 | Shaking | 99.15 | 0.70 | 0.15 | 0.019 | 1.04 | No |
| | for 5 days | | | | | | particles, |
| | at 150 rpm | | | | | | clear |
| | and RT | | | | | | |
| Example 3 | 3 freeze/ | 99.75 | 0.14 | 0.12 | 0.018 | 1.00 | No |
| | thaw | | | | | | particles, |
| | cycles | | | | | | clear |
| | between | | | | | | |
| | −20° C. and | | | | | | |
| | +5° C. | | | | | | |

The results clearly show that the formulation according to the invention has significantly increased stability compared with the comparative solutions of the prior art.

The entire disclosures of all applications, patents and publications, cited herein and of corresponding German application No. 1035521.0, filed Nov. 26, 2003, and U.S. Provisional Application Ser. No. 60/543,287, filed Feb. 11, 2004 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Aqueous preparation comprising an anti-EGFR antibody, a buffer, an amino acid and a surfactant.

2. Preparation according to claim 1, characterised in that the antibody is cetuximab or EMD 72000 or one of the corresponding murine, humanised or chimeric antibody analogues.

3. Preparation according to claim 2, characterised in that the antibody is cetuximab or EMD 72000.

4. Preparation according to claim 1, characterised in that the buffer consists of one or more citrate salt(s), acetate salt(s), histidine salt(s), succinate salt(s), malate salt(s), phosphate salt(s) or lactate salt(s) and/or the respective free acid(s) or base(s) thereof or a mixture of one or more of the various salts and/or the acid(s) or base(s) thereof.

5. Preparation according to claim 4, characterised in that the buffer consists of one or more citrate salt(s) and/or the free acid thereof, acetate salt(s) and/or the free acid thereof or L-histidine and/or an acid-addition salt thereof.

6. Preparation according to claim 1, characterised in that the amino acid is L-arginine, glycine or L-methionine.

7. Preparation according to claim 1, characterised in that the surfactant is a polyethylene sorbitan fatty acid ester or a polyoxyethylene-polyoxypropylene copolymer.

8. Preparation according to claim 7, characterised in that the polyoxyethylene sorbitan fatty acid ester surfactant is polyoxyethylene (20) sorbitan monooleate or polyoxyethylene (20) sorbitan monolaurate.

9. Preparation according to claim 7, characterised in that the surfactant is Poloxamer 407.

10. Preparation according to claim 1, characterised in that an isotonicity modifier is furthermore present in a concentration necessary for isotonicity modification.

11. Preparation according to claim 10, characterised in that the isotonicity modifier is sodium chloride.

12. Preparation according to claim 1, characterised in that it has a pH of 5-7, preferably from pH 5.2 to pH 6.0.

13. Preparation according to claim 12, characterised in that it has a pH of about 5.5.

14. Preparation according to claim 1, characterised in that it comprises about 5 mg/ml of cetuximab or EMD 72000, about 10 mmol/l of citrate or histidine buffer, about 100 mmol/lof glycine, L-arginine or L-methionine, about 100 mmol/l of sodium chloride and about 0.01% of polyoxyethylene (20) sorbitan monooleate and has a pH of about 5.5.

15. Process for the preparation of a pharmaceutical preparation according to claim 1, characterised in that an aqueous preparation comprising the anti-EGFR antibody is added to one of the said auxiliaries.

16. Use of the preparation according to claim 1 for the treatment of tumour diseases.
